(19) United States
(12) Patent Application Publication
(10) Pub. No.: US 2026/0305936 A1
(43) Pub. Date: Oct. 8, 2026

(54) SCANNING OCULAR WAVEFRONT SENSOR FOR CHARACTERIZING OPTICAL QUALITY ACROSS RETINAL ECCENTRICITY

(71) Applicants: UNIVERSITY OF HOUSTON SYSTEM, Houston, TX (US); UNIVERSITY OF ROCHESTER, Rochester, NY (US)

(72) Inventors: Geunyoung YOON, Houston, TX (US); Chloe DEGRE, Houston, TX (US); Yifei WU, Cumming, GA (US)

(73) Assignees: UNIVERSITY OF HOUSTON SYSTEM, Houston, TX (US); UNIVERSITY OF ROCHESTER, Rochester, NY (US)

(21) Appl. No.: 19/478,890

(22) PCT Filed: May 2, 2024
(86) PCT No.: PCT/US2024/027450

§ 371 Date: Oct. 27, 2025

Related U.S. Application Data
(60) Provisional application No. 63/463,484, filed on May 2, 2023.

(51) Int. Cl. A61B 3/10; A61B 3/14

(52) U.S. Cl. CPC A61B 3/1015; A61B 3/1025; A61B 3/14

(57) ABSTRACT

An imaging system for capturing images of the periphery of an eye of a patient is disclosed. An optical signal source provides an optical source light. A microlens array is placed at the pupil conjugate of the patient. An image sensor receives focused light from the microlens array. A horizontal scan system is coupled to the microlens array and the image sensor to capture wavefront images in a scan range in the horizontal plane to the eye. A vertical scan system is coupled to the microlens array and the image sensor to capture wavefront images in a scan range in a vertical plane to the eye. A controller controls the horizontal scan system and the vertical scan system. The horizontal scan system captures images in a horizontal scan. The vertical scan system captures images in a vertical scan. Both the horizontal scan system and the vertical scan system are activated to capture images in a diagonal scan.

### PRIORITY CLAIM

The present disclosure claims the benefit of and priority to U.S. Provisional Application Ser. No. 63/463,484 filed May 2, 2023. The contents of that application are hereby incorporated by reference in their entirety.

### STATEMENT REGARDING FEDERALLY SPONSORED RESEARCH OR DEVELOPMENT

This invention was made with government support under Grant No. EY034151 awarded by the National Institutes of Health. The government has certain rights in the invention.

### TECHNICAL FIELD

The present invention relates generally to a scanner instrument for an eye. More specifically, a scanning ocular wavefront sensor instrument having multi-dimensional control for peripheral scanning is disclosed.

### BACKGROUND

Ocular wavefront sensing technology has been a game changer in the field of ophthalmology and optometry (refractive and cataract surgery, and contact lens) over the past two decades. However, the applications of the sensing technology have been limited to the central retina (fovea). A new emerging need for this technology is the treatment of myopia which is a global public health concern that is expected to affect 50% of the world's population by 2050. Among many theories for cause of myopia development/progression, one of the most popular ones is the peripheral optics of the eye.

Myopia is a global concern, affecting more than 30% of the world's population and up to an alarming rate of 92% of the youth population in south and east Asian countries. Besides a substantial socio-economic burden, high myopia increases the risk of blindness through associated ocular pathologies like retinal disorders, glaucoma, and cataract. Acknowledging the significant need for myopia control, recent studies have widely discussed relative peripheral refraction (RPR) as a critical parameter to comprehend the underlying causes of myopia progression and to develop prevention strategies. The study of peripheral refraction came to the limelight in 1971 when RPR profiles were used to predict future myopia incidence in young pilots. The important contribution of the peripheral retina to visual system development was established by an animal study, which showed emmetropization can continue even after central retinal laser ablation. Studies conducted through cross-sectional research have suggested that myopic eyes are more likely to experience relative hyperopic peripheral defocus, while emmetropic and hyperopic eyes tend to have a relatively myopic periphery. Further investigations in animal models and human eyes supported the hypothesis that peripheral hyperopic defocus may be responsible for retinal elongation or the development of axial myopia. On the contrary, recent longitudinal studies in children predicted that relative peripheral hyperopia may have little or no influence on the risk of myopia development. Therefore, it is an ongoing debate whether the relative difference in peripheral refraction is a consequence or cause of myopia development. Irrespective of this debate, many scientists and industries have gained interest in myopia control which has led to the development of corrective lenses that are designed to add plus power to the periphery with the goal of shifting relative peripheral refraction to be more myopic.

While these studies bring attention to the interaction between optically induced myopia and the peripheral retina, it is important to recognize that peripheral ocular optics are more complex than just defocus. Unlike the defocus-dominated central retina, the peripheral ocular optics are primarily affected by astigmatism and non-rotationally symmetric higher-order aberrations (HOAs) like comas. These complex aberrations result in an anisotropic or directional optical blur. Therefore, myopia control lenses primarily designed to alter peripheral defocus also induce those asymmetric aberrations in the periphery, which then interact with the eye's native peripheral astigmatism and HOAs. However, it is not well understood how the optical properties and retinal image quality of the eye across the visual field are altered by the aberrations induced by myopia control lenses when combined with the eye's native aberrations.

Evaluation of peripheral ocular optics is also important as it can affect visual performance in individuals with normal or compromised central vision. The impact of peripheral aberrations on daily activities such as reading, driving, walking, and playing sports is also of great interest. In addition, research has explored peripheral aberrations in individuals with pseudophakia or keratoconus, as well as how peripheral optics influence contrast sensitivity, accommodation, and pupil size variation. Overall, there is a growing body of research that highlights the importance of evaluating peripheral ocular optics. Consequently, the creation of an optical tool capable of measuring peripheral eye aberrations is crucial to conducting both basic and clinical research, particularly in comprehending myopia mechanisms and designing optical tactics to prevent and decelerate its progression.

In the last decades, several approaches have been developed to measure peripheral ocular optics with Shack-Hartmann (SH) wavefront sensors. Currently, several methods employ either continuous or discrete point scanning techniques. While current peripheral wavefront scanning sensors are an exciting development in this field, they still have some limitations such as relatively small scanning ranges and limited scanning meridians. Additionally, some systems require either eccentric fixation through an eye or a head turn to measure the peripheral aberrations.

Recently, there has been a significant focus on the study of peripheral aberrations, particularly in the area of myopia. Peripheral aberration measurement devices have been previously attempted using several approaches. A double-pass optical system has been incorporated in a scanning SH aberrometer. Scanning in this system was achieved using two scanning mirrors, which redirect the light beam to 37 discrete measurement points located in a grid of ±15° visual field within 7 seconds. This setup allows the instrument to use primary gaze fixation during wavefront measurement, however, measurements are limited to a relatively small field. An alternate approach was used in the BHVI-EyeMapper, employing an SH wavefront sensor that features a complex system of 33 fixed mirrors and one scanning mirror. This sensor can measure the horizontal retinal eccentricity up to ±50° in increments of 10 degrees within 0.5 seconds while maintaining central fixation. However, while this sensor is speedy and can cover a wide field, it is limited to discrete retinal locations only along the horizontal axis. Another SH peripheral wavefront sensor with a wide-field horizontal scanning capability can scan±40° horizontally and provides 81 high-resolution images in 1.8 seconds. This scanning device is fast and capable to measure aberration at every 1° interval. However, this scanning device is limited to a horizontal scan only. Thus, limitations such as a smaller scanning range, limited to one meridian, or requiring eccentric fixation through ocular rotation or head turn restrict current instruments.

Thus, there is a need for a robust scanning wavefront sensor equipped with a scanning mechanism with wide scanning ranges and scanning meridians for analyzing aberrations in a pupil. There is also a need for a scanning instrument that allows precise control of the scanner over different dimensional ranges. There is a further need for a scanner that allows rapid ocular measurements. There is also a need for developing a diagnostic tool for characterizing peripheral optics via wavefront sensors to allow for optical intervention to control myopia progression.

### SUMMARY

One disclosed example is an imaging system for capturing images of the periphery of an eye of a patient. The system includes an optical signal source providing an optical source light. A microlens array is placed at the pupil conjugate of the patient. An image sensor receives focused light from the microlens array. A horizontal scan system is coupled to the microlens array and the image sensor to capture images in a scan range of angles in the horizontal plane to the eye. A vertical scan system is coupled to the microlens array and the image sensor to capture images in a scan range of vertical angles relative to a horizontal plane to the eye. A controller is coupled to the horizontal scan system and the vertical scan system. The controller activates the horizontal scan system to capture images of the eye in a horizontal scan. The controller activates the vertical scan system to capture images of the eye in a vertical scan. The controller activates both the horizontal scan system and the vertical scan system to capture images of the eye in a diagonal scan.

A further implementation of the example system is an embodiment where the horizontal scan system includes a rotational table supporting the micro-lens array and image sensor, the rotational table rotated by a first actuator. Another implementation is where the horizontal scan system includes a rotatable hot mirror directing the optical source light to the eye, a first linear actuator moving in a first dimension, and a second linear actuator coupled to the first linear actuator moving in a second dimension perpendicular to the first dimension. The micro-lens array and image sensor are coupled to the second linear actuator. The first linear actuator, the second linear actuator, and the rotatable hot mirror are controlled in the scan range of angles in the horizontal plane. Another implementation is where the vertical scan system includes a hot mirror directing the optical source light to the eye, a first linear actuator moving in a first dimension, and a second linear actuator coupled to the first linear actuator moving in a second dimension perpendicular to the first dimension, wherein the micro-lens array and image sensor are coupled to the second linear actuator, wherein the first linear actuator, the second linear actuator and the rotatable hot mirror are controlled in the scan range of angles in vertical horizontal plane. Another implementation is where the hot mirror is rotated via a belt coupled to a motor. Another implementation is where the patient maintains foveal fixation on the target object through the hot mirror, and the hot mirror is a rectangular shape. Another implementation is where the example system includes a goniometer coupled to hot mirror to rotate the hot mirror. Another implementation is where the example system includes a target object and a cold mirror allowing the patient to focus on the target object. Another implementation is where the vertical scan system is a goniometer. The goniometer swings the micro-lens array and wavefront image sensor in the vertical plane relative to the eye. Another implementation is where the image sensor is activated by the controller to provide real-time images at predetermined degree ranges in the horizontal, vertical and diagonal retinal eccentricities. Another implementation is where the image sensor takes a plurality of images along predetermined degree increments in the horizontal, vertical, and diagonal ranges. Another implementation is where the imaging sensor is a high sensitivity charge coupled device (CCD) sensor. Another implementation is where the imaging sensor is a Shack-Harmann wavefront imaging sensor. Another implementation is where the example system includes an output beam splitter receiving the reflected light from the eye through the microlens array and splitting the reflected light to the imaging sensor. Another implementation is where the example system includes a pupil camera accepting the reflected light from the output beam splitter to monitor eye movements of the patient. Another implementation is where the optical source is a laser diode. Another implementation is where the example system includes a ring illuminator positioned in proximity to the eye. The pupil is illuminated via the ring illuminator.

Another disclosed example is an imaging system for capturing images of the periphery of an eye of a patient. The system includes an optical signal source providing an optical source light. A microlens array is placed at the pupil conjugate of the patient. A wavefront image sensor receives focused light from the microlens array. A rotational table is coupled to the microlens array and the wavefront image sensor to rotate the microlens array and the wavefront image sensor capture wavefront images in a scan range of angles in the horizontal plane to the eye. A vertical scan system is mounted on the rotational table to capture wavefront images in a scan range of vertical angles relative to a horizontal plane to the eye. The vertical scan system includes a hot mirror, a goniometer supporting the hot mirror to direct the optical source light to the eye, a first linear actuator moving in a first dimension, and a second linear actuator coupled to the first linear actuator moving in a second dimension perpendicular to the first dimension. The goniometer, the micro-lens array and wavefront image sensor are coupled to the second linear actuator. The first linear actuator, the second linear actuator, and the goniometer are controlled in the scan range of angles in vertical horizontal plane. A controller is coupled to the horizontal scan system and the vertical scan system. The controller activating the horizontal scan system to capture wavefront images of the eye in a horizontal scan. The controller activates the vertical scan system to capture wavefront images of the eye in a vertical scan. The controller activates both the horizontal scan system and the vertical scan system to capture wavefront images of the eye in a diagonal scan.

Another disclosed example is a method for capturing images for detection of aberrations in an eye. An optical source light is provided at the pupil conjugate of the eye. Images of the eye are captured via a microlens array over a horizontal range of angles in the horizontal plane to the eye by positioning an image sensor coupled to a microlens array via a horizontal scan system over the horizontal range. Images of the eye are captured via the microlens array over a vertical range of angles in the horizontal plane to the eye by positioning the image sensor coupled to the microlens array via a vertical scan system over the vertical range. Images of the eye are captured via the microlens array over a diagonal range of angles in the horizontal plane to the eye by positioning the image sensor coupled to the microlens array via both the horizontal scan system and the vertical scan system over the diagonal range.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of embodiments together with reference to the accompanying drawings.

FIG. 1 is a block diagram of an example image scanning assembly for capturing images and Shack-Hartmann (SH) wavefronts of a pupil;

FIG. 2A is a perspective view of an example scanning system incorporating the image scanning assembly in FIG. 1;

FIG. 2B is a perspective view of the example scanning system in FIG. 2A used for capturing scans of the eye of a patient;

FIG. 3 is a block diagram of the control system of the scanning system in FIG. 2A;

FIG. 4 is a block diagram of another example image scanning assembly for capturing images and Shack-Hartmann (SH) wavefronts of a pupil;

FIG. 5 is a perspective view of another scanning system incorporating the image scanning assembly in FIG. 4;

FIG. 6 is a block diagram of the control system of the scanning system in FIG. 5;

FIG. 7A is a perspective view of the scanning system in FIG. 5 rotated at one end of the horizontal scan range;

FIG. 7B is a perspective view of the scanning system in FIG. 5 rotated at the opposite end of the horizontal scan range;

FIG. 7C is a perspective view of the scanning system in FIG. 5 rotated at the center of the horizontal scan range;

FIG. 8A is a perspective view of the scanning system in FIG. 5 positioned at one end of the vertical scan range;

FIG. 8B is a perspective view of the scanning system in FIG. 5 positioned at the opposite end of the vertical scan range;

FIG. 8C is a perspective view of the scanning system in FIG. 5 positioned at the center of the vertical scan range;

FIG. 9A is a perspective view of another scanning system incorporating the image scanning assembly in FIG. 4;

FIG. 9B is a side view of the scanning system in FIG. 9A;

FIG. 10 a block diagram of the control system of the scanning system in FIG. 9A;

FIG. 11A is a perspective view of the scanning system in FIG. 9A positioned at one end of the horizontal scan range;

FIG. 11B is a perspective view of the scanning system in FIG. 9A positioned at the opposite end of the horizontal scan range;

FIG. 11C is a perspective view of the scanning system in FIG. 9A positioned at the center of the horizontal scan range;

FIG. 12A is a perspective view of the scanning system in FIG. 9A positioned at one end of the vertical scan range;

FIG. 12B is a perspective view of the scanning system in FIG. 9A positioned at the opposite end of the vertical scan range;

FIG. 12C is a perspective view of the scanning system in FIG. 9A positioned at the center of the vertical scan range;

FIG. 13 is a side view of another scanning system incorporating the image capture assembly in FIG. 4;

FIG. 14 is a block diagram of the control system of the scanning system in FIG. 10A;

FIG. 15 is a flow diagram of the control system for the example scanning systems;

FIG. 16 is a series of graphs of data measured from the example scanning systems compared with model eye data;

FIG. 17 shows a series of graphs that show peripheral lower-order aberrations (LOAs) plotting Zernike coefficient against retinal eccentricity;

FIG. 18 shows a series of graphs that show higher-order aberrations (HOAs) across the retinal eccentricity;

FIG. 19 shows plots of pan-retinal schematic representations of wavefront maps reconstructed from the measured aberrations across all four scanning directions; and

FIG. 20 is a graph that shows relative defocus (D) from the fovea across horizontal meridian in myopic and non-myopic groups.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The example disclosure relates to a wide field scanning wavefront sensor (WSWS) device with multidirectional scanning capability for an eye, while maintaining natural eye fixation at primary gaze. The example sensor device includes vertical and horizontal scan movement systems that allow image captures of a pupil across a horizontal range, a vertical range, and a diagonal range. The example sensor device includes a wavefront sensor that may be positioned by the vertical and horizontal scan movement systems to scan any area of the retina across a wide range with multi-directional capability. The example system is optimized for natural eye fixation at the primary gaze, eliminating the requirement for eccentric fixation and head turn during peripheral aberration measurement. The example instrument allows open view and foveal fixation that allows controlling accommodation precisely, resulting in avoiding artifacts in the measurement. The resulting wavefront images may be analyzed to determine aberrations. The open-view natural fixation mechanism improves measuring peripheral optics with and without an optical myopia intervention, such as a contact lens in both children and adults, particularly in studying myopia. The performance has been tested and demonstrated precise peripheral measurement of both LOAs and HOAs.

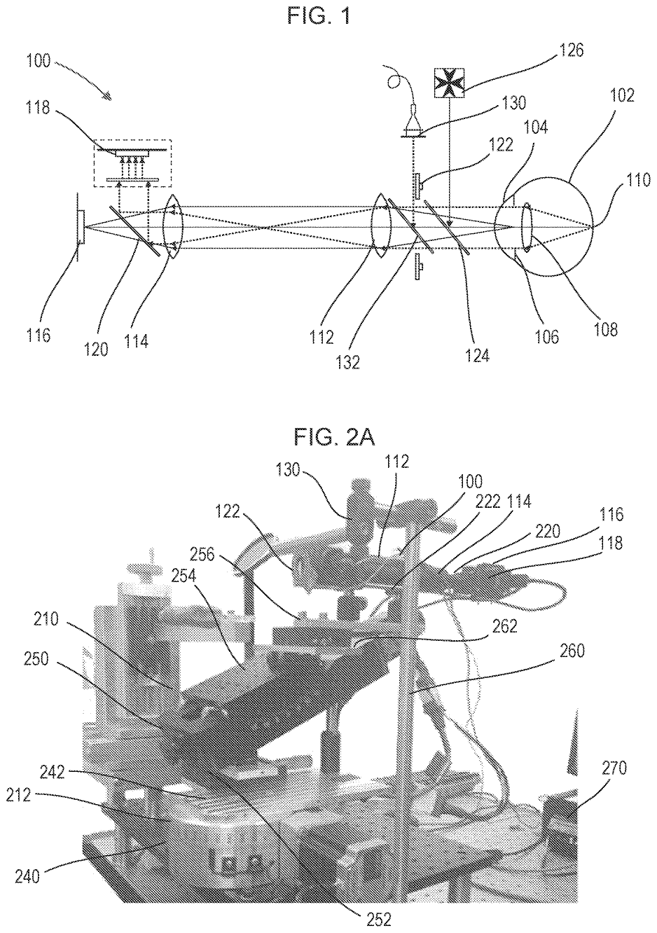

FIG. 1 is a block diagram of an example widefield scanning assembly 100. The scanning assembly 100 is designed to capture images of an eye 102 in different scan dimensions. The eye 102 includes an iris 104, a pupil 106, a lens 108, and a retina 110. Images projected through the pupil 106 and the lens 108 on the retina 110 are captured through two lens 112 and 114. As will be explained, example scanner devices are disclosed that actuate the scanning assembly 100 over different orientations relative to the eye 110.

A pupil camera 116 receives images of the retina 110 from the lenses 112 and 114. A wavefront sensor 118 receives images from the retina 110 through the pupil 106 via a dichroic beam splitter 120 that splits the beam from the lens 114 between the wavefront sensor 118 and the camera 116. The patient positions their eye 102 through a ring LED 122 that serves to illuminate the pupil 106. A cold mirror 124 reflects an image of a target object such as a Maltese cross 126 to the pupil 106. A beam splitter 128 deflects a laser signal emitted by a laser 130 toward the pupil 106.

The example scanning assembly 100 is designed to measure ocular aberrations at various eccentricities across any meridian of the eye 102. The scanning assembly 100 includes both illumination and measuring optics. The laser 130 in this example is an 850 nm laser diode source in this example that has a 400 μm pinhole for focus the laser signal. The laser power entering the eye is 40 μW, which is over ten times lower than the ANSI national laser safety standard. The near-infrared light is directed toward the pellicle beam-splitter (BS1) 128 that is oriented at 45-degree-oriented to reflect the light entering the eye. The reflected backlight from the retina passes through the beam splitter 128 through a 4-F telescopic system. In this example, a 4-F telescopic system includes the lens 112 and the lens 114. The lens 112 has f=100 mm and the lens 114 has f=75 mm, resulting in 25% pupil demagnification. The transmitted light is then reflected by another beam-splitter (BS2) 120 and enters the SH wavefront sensor 118. In this example, the wavefront sensor 118 uses a micro-lenslet array with a pitch of 300 μm and 4.5 mm focal length placed at the pupil conjugate. In this example, two identical CMOS sensors (MQ013RG-e2, Ximea, Inc.) are used for the wavefront sensor 118 and the pupil imaging camera 116. The sensors have a resolution of 1280×1024 pixels, an active sensor dimension of 6.9×5.5 mm (pixel size: 5.4 μm), and high sensitivity for the near-infrared range. The ring LED 122 has a wavelength 950 nm and illuminates the pupil for alignment of the pupil center to the optical axis of the system, and the beam splitter 120 BS2 with a cutoff wavelength of 900 nm. The ring LED 122 separates the pupil illumination from the wavefront sensing light. The Maltese cross distance fixation target 126 is co-aligned with the optical system of the instrument and allows for steady fixation. The cold mirror 124 in this example is relatively large (2 inches) redirects the target Maltese cross to the primary gaze fixation of the patient. A lens can be inserted into the open-view fixation optical path to correct for subjective refractive error and to control accommodation. As will be explained, the example scanning assembly 100 allows capture of retina images and wavefront images from numerous ranges of horizontal and vertical directions based on coordinated control of horizontal and vertical actuation assemblies that move the mirrors and detectors in FIG. 1.

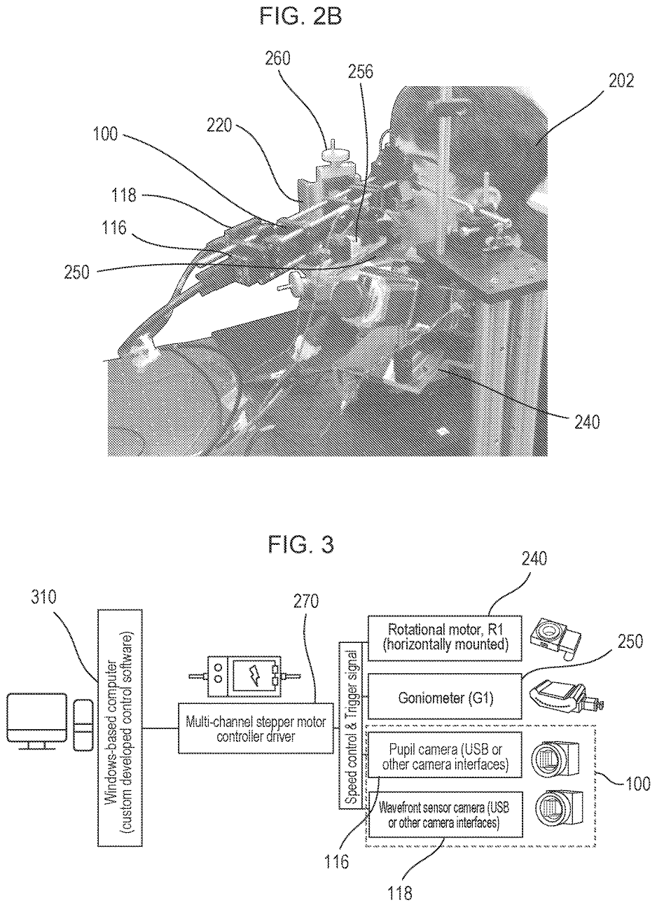

An example scanning instrument 200 is shown in FIG. 2A that includes the scanning assembly 100 for capturing images in FIG. 1. FIG. 2B is a perspective view of the example scanning instrument 200 in position to take scans from the eye of a patient 202. The scanning instrument 200 includes a vertical scan movement system 210 and a horizontal scan movement system 212 to allow the example scanning instrument 200 to scan the periphery of the eye of the patient 202.

The vertical and horizontal scan movement systems 210 and 212 allow the scanning instrument 200 to capture images along a horizontal range, a vertical range, and a diagonal range. The vertical movement system 210 includes a telescopic assembly 220 having a series of support rods 222 that holds various components of the scanning assembly 100 in FIG. 1. Thus, the telescopic assembly 220 holds the optical signal source of the laser 130 providing an optical source light. The telescopic assembly 220 holds the input beam splitter directing the optical source light toward the eye of the patient 202. In this example, a microlens array of the lens 112 and 114 is supported by the telescopic assembly 220 to be placed at the pupil conjugate of the patient. One end of the telescopic assembly 220 holds the ring illuminator 122. The other end of the telescopic assembly 220 holds the output beam splitter receiving the reflected light from the eye through the microlens array. The other end of the assembly 220 also includes the wavefront image sensor 118 receiving focused light from the microlens array and the pupil imaging camera 116, which is a CCD image sensor. A fixation unit (not shown) including the cold mirror 124 and the Maltese cross 126 in FIG. 1 is positioned at a set distance from the eye of the patient 202. The fixation unit may have a moveable support that allows the cross 126 to be positioned at different distances from eye of the patient 202.

The horizontal scan system 212 is driven by an actuator such as a motor that rotates a rotational stage 240. The horizontal scan system 212 is moveable by the motor of the rotational stage 240 to allow the scanning assembly 100 to capture wavefront images in a scan range of angles in the horizontal plane to the eye of the patient 202. The rotational stage 240 is attached to a lateral support plate 242.

The vertical scan system 210 includes an actuator which in this example is a goniometer 250 including a stepping motor 252 that is mounted to the lateral support plate 242 rotated by the rotational stage 240. The goniometer 250 is mounted to the rotational stage 240 with its rotational axis perpendicular to the rotational axis of the rotational stage 240. The goniometer 250 includes a rotating arm member 254 that is attached to a plate 256 supporting the telescopic assembly 220. The goniometer 250 on the rotational stage 240 thus positions the telescopic assembly 220 and the components of the scanning assembly 100 at the eye level of the patient 202.

A three axis bite bar assembly 260 allows the patient 202 to position the chin relative to the scanning assembly 100 by placing the chin on an extended arm 262. A controller 270 is coupled to the vertical scan system 210 and the horizontal scan system 212. In this example, the controller is a Velmex VXM type motor controller, but any suitable motor controller may be used. The controller 270 activates the first rotational actuator (the rotational stage 240) to rotate the horizontal scan system 212. The controller 270 controls the scanning assembly 100 to capture wavefront images of the eye in a horizontal scan range at intervals along the scan range. The controller activates the goniometer 250 to move the scanning assembly 100 to capture wavefront images of the eye at intervals in a vertical scan range. The controller activates both the motor of the rotational stage 240 and the goniometer 250 in concert and activates the scanning assembly 100 to capture wavefront images of the eye in a diagonal scan range. In this example, the scanning routine controlling the rotational stage 240, the goniometer 250, the CMOS wave front sensor 118, and pupil image sensor 116 are synchronized and controlled by custom-built software based on C++ that is executed by an external computing device.

In order to facilitate scanning along any meridian, the complete optical arm 220 is affixed to a rotational stage 240 via the goniometer 250. In this example, the rotational stage is a 4800 series available from Velmex Inc., USA. The cold mirror 124 and the Maltese cross 126 in FIG. 1 are kept stationary on the fixation unit relative to the other components of the instrument 200 to preserve central eye fixation. In this example, the high-capacity stage of the rotational stage 240 has an accuracy of 100 arc-seconds and repeatability of 1 arc-second. The motor 252 of the goniometer 250 in this example is a Y07is-59D1-17155 motor from Shinano Kenshi Co. Ltd, Japan. As explained above, in this example, the goniometer 250 controls the vertical rotation of the scanning assembly 100 while the rotational stage 240 controls the horizontal rotation of the scanning assembly 100. The centers of rotation (CoR) of the motor driving the rotational stage 240 and the motor 252 of the goniometer 250 are co-aligned to the center of the pupil of the eye of the patient 202.

FIG. 3 shows the control system of the scanning instrument 200 in FIG. 2A. In this example, operation of individual motors of the rotational stage 240 and the goniometer 250 by the controller 270 allows for horizontal and vertical scanning, while any diagonal meridian scan can be achieved by precisely synchronizing and combining the movement of both motors. In this example, the control signals are generated by a personal computer 310 with the custom-built control software. The personal computer 310 is coupled to the controller 270 that is a multi-channel stepper motor controller driver. The controller 270 provides trigger signals to the motors of the rotational stage 240 and the goniometer 250 to adjust the angle of each for the scan patterns. Over each of the ranges, the controller 270 activates the image sensor 116 and the SH wavefront sensor 118 to capture sensed data from the eye at different positions over the scan ranges.

In this example, 45° and 135° meridians are scanned by moving both the rotational stage 240 and the goniometer 250 at a similar speed, however, running one of the motors at a higher or lower speed compared to the other motor allows for scanning of any other diagonal meridian. The pupil plane of the example instrument 200 is aligned to scan along four meridians: ±30° horizontally, ±18° vertically, and ±18° obliquely along the 45° and 135° meridians. The vertical and oblique scans are limited mainly by the obstruction of the incoming laser beam at extreme eccentricities by the anatomical structures around the eye (i.e. the eye socket and forehead), and the large size of the goniometer 250.

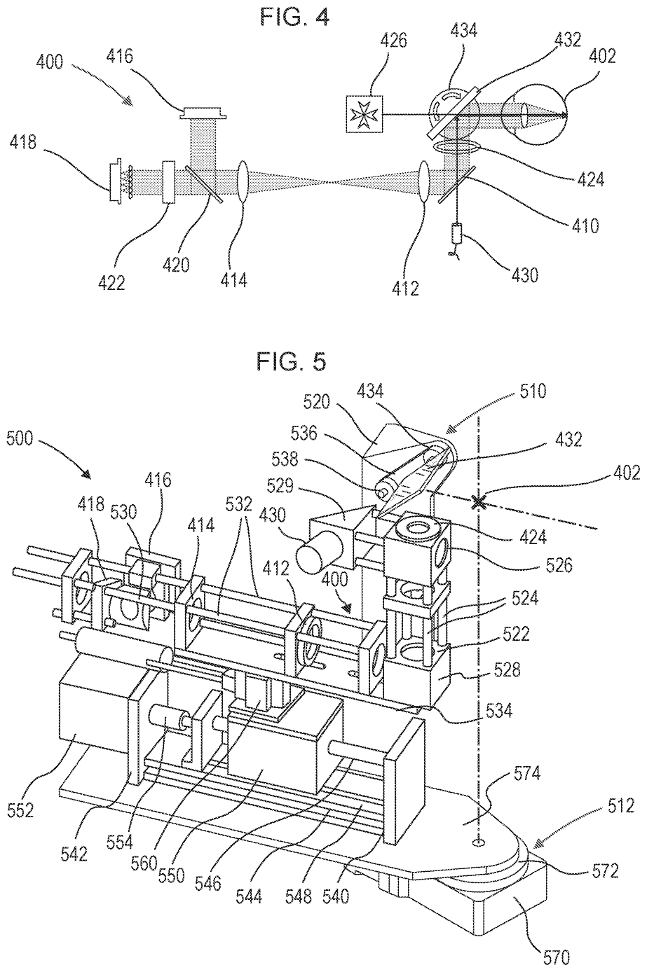

FIG. 4 is a block diagram of another example widefield scanning assembly 400. The scanning assembly 400 is designed to capture images of an eye 402 of a patient. Images projected through the pupil and the lens on the retina of the eye 402 are deflected by a folding mirror 410 and captured through two lens 412 and 414.

An imaging sensor such as a pupil camera 416 receives images of the retina of the eye 402 from the lens 412 and 414. A wavefront sensor 418 receives images from the retina via a dichroic beam splitter filter 420 that splits the beam from the lens 414 between the wavefront sensor 418 and the camera 416. A long pass filter 422 filters the beam that is directed to the wavefront sensor 418. The patient positions their eye 402 through a ring LED 424 that serves to illuminate the pupil. A target object such as a Maltese cross 426 is viewed by the eye 402.

In this example, a laser 430 emits a laser signal that is deflected by a rotating hot mirror 432 that directs the beam to illuminate the eye 402. As will be explained, the hot mirror 432 is mounted on a drive wheel 434 that is rotated by a motor.

FIG. 5 is a perspective view of another example scanning instrument system 500 that allows the positioning of the scanning assembly 400 in FIG. 4 relative to the eye 402. The scanning instrument 500 includes a vertical scan movement system 510 and a horizontal scan movement system 512 to allow the example scanning instrument 500 to scan the periphery of the eye of the patient 402 represented by an x symbol.

The vertical and horizontal scan movement systems 510 and 512 allow the scanning system 500 to capture images of the eye 402 along a horizontal range, a vertical range, and a diagonal range. The vertical scan movement system 510 includes an optical arm 520 that holds a vertical cage assembly 522 that includes a series of rods 524 that support a beam splitter compartment 526 and a folding mirror compartment 528. The beam splitter compartment 526 supports a laser cage assembly 529 that holds the laser 430 in FIG. 4. The laser 430 emits the beam that is directed by a beam splitter in the beam splitter compartment 526 to the hot mirror 432. The reflected image is transmitted through the cage assembly 522 to the folding mirror 410 in the folding mirror compartment 528. A fixation unit (not shown) positions the Maltese cross 426 in FIG. 4 in front of the patient 402.

The lens 412 and 414 and the sensors 416 and 418 of the scanning assembly 400 are supported by a horizontal cage assembly 530 that includes a series of rods 532 that support the components. The horizontal cage assembly 530 includes a longitudinal plate 534 that supports the components of the scanning assembly 400 as well as the optical arm 520 and the vertical cage assembly 522. The optical arm 520 holds the axle of the drive wheel 434 that rotates the hot mirror 432. The drive wheel 434 is rotated by a belt 536 that is looped around a motor wheel 538 that is driven by a motor supported by the optical arm 520. The motor rotates the motor wheel 538 and thus the hot mirror 432.

In this example, a microlens array of the lenses 412 and 414 is supported by the support rods 532 and the longitudinal plate 534 of the horizontal cage assembly 530. The other end of the horizontal cage assembly 530 holds the output beam splitter 420 receiving the reflected light from the eye 402 through the microlens array. The other end of the horizontal cage assembly 530 also includes the wavefront image sensor 418 receiving focused light from the microlens array and the CCD image sensor 416.

A longitudinal linear position system includes a pair of vertical blocks 540 and 542 that support a stage 544 and a transversal rod 546. The top surface of the stage 544 includes a track 548 that allows a trolley 550 driven by a motor 552. The motor 552 rotates a rotating screw rod 554 that creates linear motion to traverse the trolley 550 on the track 548 and the rod 546. The trolley 550 is mechanically coupled to the scanning assembly 400 and allows movement in a z-axis relative to the mirror 432. The longitudinal plate 534 of the horizontal cage assembly 530 is supported by a vertical actuator 560 that moves the scanning assembly 400 up and down. As will be explained, as the hot mirror 432 is angled by the motor in the optical arm 520, the longitudinal plate 534 may be moved on the z-axis by the trolley 550 and in the y-axis by the vertical actuator 560 to position the scanning assembly 400 relative to the mirror 432 to capture the scans at a vertical angle range.

The horizontal scan system 512 includes a base 570 that holds an actuator, which in this example is a motor that rotates a circular base 572. The circular base 572 is attached to a support plate 574. The horizontal scan system 512 is moveable by the motor to allow the scanning assembly 400 to capture wavefront images in a scan range of angles in the horizontal plane to the eye of the patient. The support plate 574 supports the longitudinal linear position system.

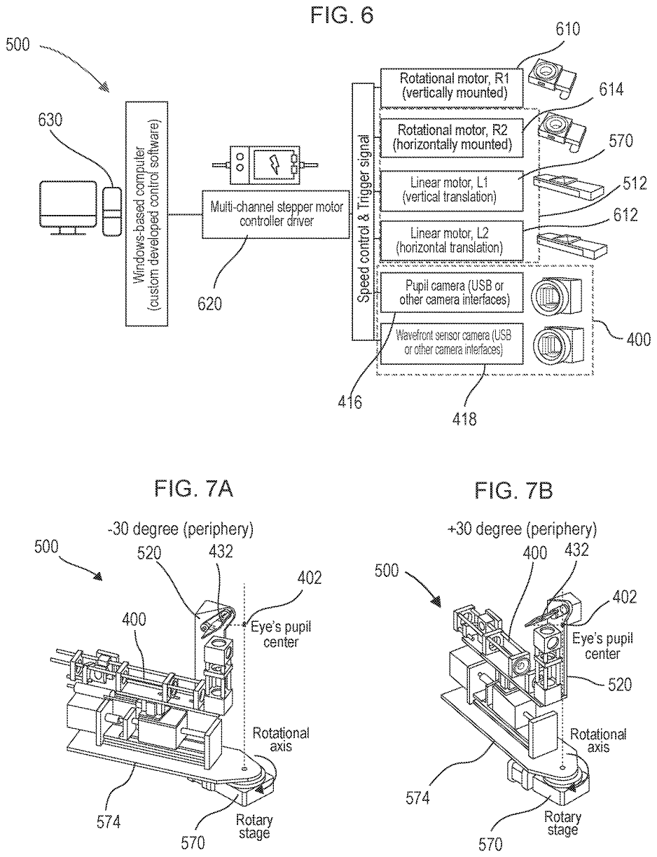

FIG. 6 is a block diagram of the control system for the scanning system 500 in FIG. 5. In this example, the horizontal scan system 512 includes a motor 610 in the base 570 of the rotational table. The vertical scan system 510 includes a longitudinal linear actuator system 612 that includes the trolley 550 that transverses the track 548 in FIG. 5, the vertical actuator 560, and a motor 614 that rotates the mirror 432 in FIG. 5. A controller 620 generates commands for operation of individual motor 610 of the rotational table that rotates the circular base 572 and support plate 574, the motor driving the linear actuator system 612, the motor of the vertical actuator 560, and the motor 614 that rotates the mirror 432. In this example, the controller 620 may be a RSW Series controller from Zaber, but any suitable motor controller may be used. Control of these motors by the controller 620 allows for horizontal and vertical scanning by the scanning assembly 400, while any diagonal meridian scan can be achieved by precisely synchronizing and combining the movement of both the horizontal scan system 512 and the vertical scan system 510. In this example, the control signals are generated by a personal computer 630 with control software. The personal computer 630 is coupled to the controller 620 that is a multi-channel stepper motor controller driver. The controller 620 provides trigger signals to the motors to adjust the angle of the scanning assembly 400 in multiple dimensions for the scan patterns. Over each of the ranges, the controller 620 activates the image sensor 416 and the SH wavefront sensor 418 to capture sensed data from the eye over the scan ranges.

The controller 620 activates the motor to rotate the base 572 and thus the scanning assembly 400 to capture wavefront images of the eye in a horizontal scan. The controller activates the motor to rotate the hot mirror 432 and move the trolley 550 and vertical actuator 560 allow the scanning assembly 400 to capture wavefront images of the eye in a vertical scan. The controller 620 sends signals to rotate the base 572, and position the trolley 550, the vertical actuator 560, and rotate the mirror 432 in concert to capture wavefront images of the eye in a diagonal scan.

The instrument 500 allows open-view fixation where the patient maintains foveal fixation through the large rectangular hot mirror 432. This eliminates instrument myopia and provides reliable control of accommodation without cycloplegia.

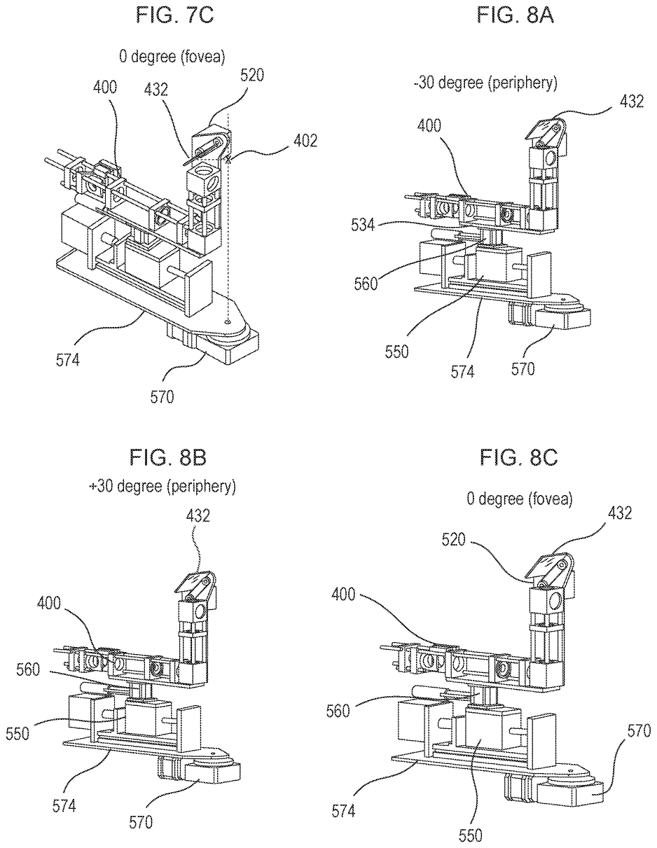

FIGS. 7A-7C shown the range of the horizontal scan through operation of the horizontal scan system 512 using the rotation of the support plate 574 relative to the base 570 to position the scanning assembly 400 and the optical support 520 holding the hot mirror 432 relative to the pupil center of the eye 402. In the horizontal scan, the hot mirror 432 is fixed a set angle 45 degrees by stopping the motor 614 at that angle. FIG. 7A shows the support plate 574 of the horizontal actuator assembly 312 rotated fully at the positive 30 degree extreme in the horizontal plane. FIG. 7B shows the support plate 574 of the horizontal actuator assembly 312 rotated fully at the negative 30 degree in the horizontal plane. FIG. 7C shows the support plate 574 of the horizontal scan system 512 rotated to the 0 degree angle of the horizontal plane. Scans may be captured by the camera 416 and the SH wave scanner 418 at any positions between the locations in FIG. 7A to FIG. 7B.

FIG. 8A-8C are perspective views of the instrument 500 that show the process of scanning along the vertical plane by the vertical scan system 510. The scan is achieved by translating the y (vertical) actuator 560 in conjunction with moving trolley 550 of the longitudinal linear actuator system 612 and rotating the hot mirror 432 around the horizontal axis. Thus, the vertical scan system 510 controls the actuator 560, the longitudinal linear actuator system 612 and the rotation of the hot mirror 432 is simultaneously to position the scanning assembly 400 at an angle in the vertical range. In this example, the hot mirror 432 is rotated at a set angle range between +10 and −10 degrees for the +20 degree to −20 degree scanning range, but other angle ranges may be used. The x linear stage actuator of the longitudinal linear actuator system 612 then moves the scanning assembly 400 in a z-axis direction along the optical axis of the pupil of the eye 402. The Y-axis linear stage actuator 560 translates the scanning assembly 100 vertically to position the laser beam emitted by the laser to intersect the optical axis of the pupil of the eye 402.

FIG. 8A shows the vertical scan at the negative 30 degree position. In this position, the longitudinal linear actuator system 612 moves the plate 534 via the trolley 550 at a position most proximal to the optical support 520. The vertical actuator 560 is lowered to a minimum height. The hot mirror 432 is rotated at a −10 degree angle position relative to the initial 45 degree position to the optical axis of the pupil of the eye 402.

The opposite extreme of the vertical range of positive 30 degrees is shown in FIG. 8B. In this position, the longitudinal linear actuator system 612 moves the plate 534 via the trolley 550 at a position most distal to the optical support 520. The vertical actuator 560 is raised to a maximum height. The hot mirror 432 is rotated at a +10 degree angle position relative to the initial 45 degree position to the optical axis of the pupil of the eye 402.

FIG. 8C shows the vertical scan assembly that allows a 0 degree position in the vertical scan range. In this position, the longitudinal linear actuator system 612 moves the plate 534 via the trolley 550 at a middle position relative to the optical support 520. The vertical actuator 560 is raised to a middle height. The hot mirror 432 is positioned at a 45 degree angle relative to the optical axis of the pupil of the eye 402.

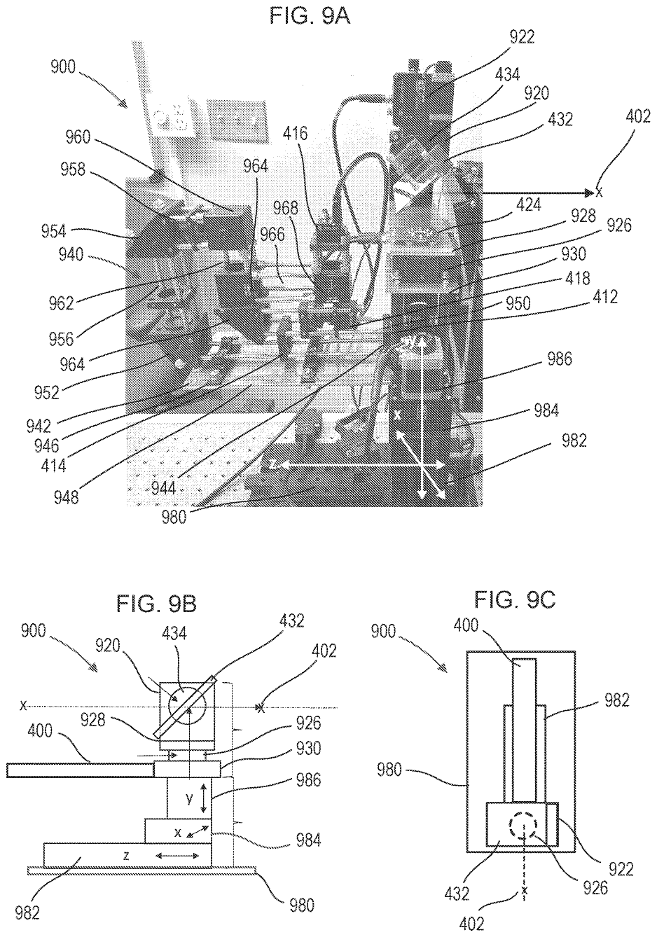

FIG. 9A is a perspective view of another example scanning instrument 900 that positions the scanning assembly 400 relative to the pupil of the patient 402. FIG. 9B is a side view of the example scanning instrument 900. FIG. 9C is a top view of the example scanning instrument 900. The scanning instrument 900 allows positioning of the scanning assembly 400 in FIG. 4 to provide vertical, horizontal and diagonal scan ranges of the eye of the patient 402 represented by an “x.”

The instrument 900 includes a vertical scan system 910 and a horizontal scan system 912. In this example, the rotary stage of the instrument 500 in FIG. 5 constituting the horizontal scan system 512 in FIG. 3A is replaced with rotating the hot mirror in combination with two linear actuator assemblies. Thus, the horizontal scan is accomplished by rotating the hot mirror using a rotational actuator in coordination with two of three linear actuators. Similar to the instrument 500, the vertical scan is accomplished by rotating the hot mirror in another plane in coordination with two of the three linear actuators. Diagonal scans involving rotating the mirror using both rotational actuators of the hot mirror and controlling all three actuators.

A mirror mounting support 920 supports the rotational mechanism for the hot mirror 432. The mirror mounting support 920 also holds a control unit 922 that controls the actuators and sensors. A first rotational actuator that includes the rotational motor driving the drive wheel 434 allows rotation around a horizontal axis as shown by the arrow in FIG. 9A. A second rotational actuator 926 has an axis of rotation perpendicular to that of the first rotational actuator driving the drive wheel 434 and thus allows rotation along a vertical axis as shown the arrow in FIG. 9A. A support plate 928 is installed on the second rotational actuator 926 and is rotated by the rotational actuator 926. The mirror mounting support 920 is attached to the support plate 928. The support plate 928 has an aperture for the laser light to be reflected from the hot mirror 432. The support plate 928 supports the ring LED 424. The second rotational actuator 926 is mounted on a lower support plate 930. In this example, the second rotational actuator 926 has a center aperture that allows the laser light to be transmitted through the aperture of the support late 928.

The other components of the scanning assembly 400 are mounted on a support assembly 940. The support assembly 940 includes a set of horizontal and vertical cages for the components of the scanning assembly 400. The support assembly 940 is thus more compact and allows the instrument 900 to require less space and less rotational movement of the extreme end of the support assembly 940 to assist in stabilization. The support assembly 940 includes a lateral plate 942 that holds a perpendicular end plate 944 and a support bracket 946. A set of lower support rods 948 are mounted between the support bracket 946 and the end plate 944. A set of corresponding upper support rods 950 are supported by the end plate 944 and a folding mirror holder 952. The lens 412 and 414 are positioned on between the rods 948 and 950.

A folding mirror structure 952 and another folding mirror structure 954 form a vertical cage 956. The folding mirror structure 954 is joined by support rods 958 to another folding mirror holder 960. The folding mirror holder 960 is attached to support rods 962 that suspend a beam splitter structure 964. The beam splitter structure 964 includes the beam splitter 420 that splits the optical signal to be directed to the wavefront sensor 418 and through a cage 966 that is mounted to another folding mirror structure 968 that diverts the optical signal to the imaging sensor 416.

The instrument 900 includes a main base 980 that allows transversal of a z-axis actuator 982 relative to the main base 980. An x-axis actuator 984 is mounted on top of the z-axis actuator 982 and traverses in an x-axis relative to the z-axis actuator 982. A y-axis actuator 986 is mounted on top of the x-axis actuator 984. The y-axis actuator 986 is attached to the lateral plate 942 of the support assembly 940. The y-axis actuator 986 also is attached and supports the lower support plate 932. Thus, the y-axis actuator 986 traverses in the y-axis.

Thus, the vertical scan system 910 includes the z-axis actuator 982, the y-axis actuator 986, and the rotational mirror actuator 924. The horizontal scan system 912 includes the z-axis actuator 982, the x-axis actuator 984, and the rotational mirror actuator 926. The mechanical stability of the entire instrument 900 is improved by the use of the three linear actuators 982, 984 and 986. The use of actuators instead of the rotational table and the compact physical structure results in lower distance of traversal and thus reduces vibrations during the scan process.

The mechanism of the horizontal scan in the instrument 900 is identical to the vertical scan mechanism in the instrument system 500. The same principle is thus applied to the horizontal plane for the instrument 900. The instrument thus includes a two-axis rotational stage (instead of one for the instrument 500 in FIG. 5) for the hot mirror 432 to control horizontal and vertical scans separately. The vertical and horizontal scans are achieved by rotating the stage via the linear actuators around the horizontal axis (pitch) and around the vertical axis (yaw).

The process for a vertical range of scans is identical to the scan process used by the z-axis actuator 612 and the vertical actuator 560 of the instrument 500 in FIG. 5. The horizontal scan uses a similar process with x-axis actuator 984 and z-axis actuator 982.

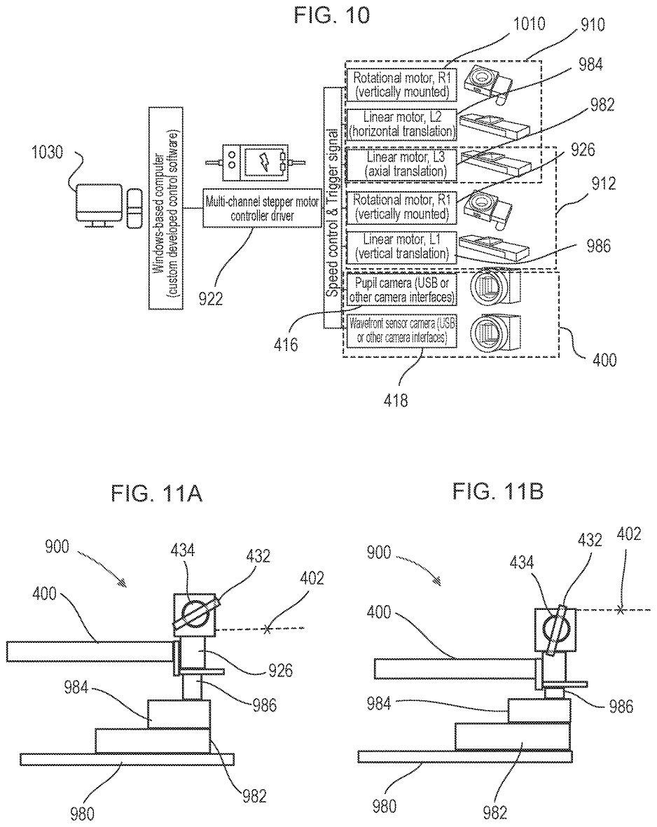

FIG. 10 shows a block diagram of the control system for the scanning system 900 in FIG. 9A. In this example, the horizontal scan system 912 includes a rotational motor 1010 that rotates the wheel 434, the x-axis actuator 984, and the z-axis actuator 982. The vertical scan system 910 includes the z-axis actuator 982, the vertical actuator 986, and the rotational actuator 926 that rotates the mirror 432. The controller 922 generates commands for operation of the individual motors in the actuators. Control of these motors by the controller 922 allows for horizontal and vertical scanning by the scanning assembly 400, while any diagonal meridian scan can be achieved by precisely synchronizing and combining the movement of both the horizontal scan system 912 and the vertical scan system 910. In this example, the control signals are generated by a personal computer 1030 with control software. The personal computer 1030 is coupled to the controller 922 that is a multi-channel stepper motor controller driver. The controller 922 provides trigger signals to the motors to adjust the angle of the scanning assembly 400 in multiple dimensions for the scan patterns. Over each of the ranges, the controller 922 activates the image sensor 416 and the SH wavefront sensor 418 to capture sensed data from the eye over the scan ranges.

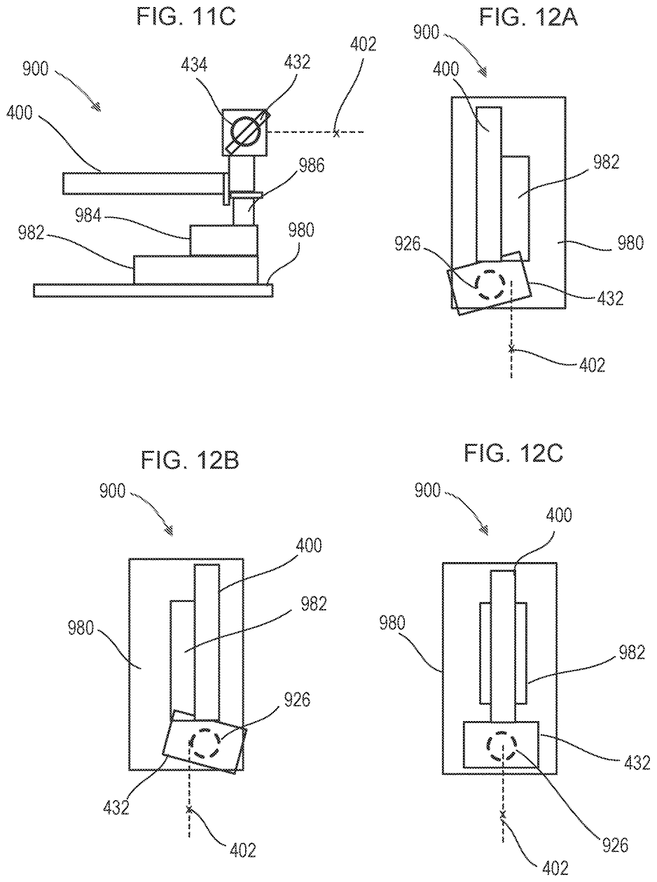

FIG. 11A shows the positions of the z-axis actuator 982 and the y-axis actuator 986 of the instrument 900 that positions the scanning assembly 400 on the positive 30 degree of the vertical range. When scanning over the vertical range, the x-axis actuator 984 is fixed in position and the z-axis actuator 982 and the y-axis actuator 986 position the scanning assembly 400 in coordination with rotating the mirror 432 with the rotational actuator rotating the drive wheel 434. Thus, the z-axis actuator 982 causes the scanning assembly 400 to be positioned at maximum distance from the pupil of the patient 402. The y-axis actuator 986 is extended to maximum height. The mirror 432 is rotated to a 55 degree angle relative to the focal plane to the eye 402.

FIG. 11B shows the negative 30 degree extreme of the vertical range of positioning the scanning assembly 400. In this position, the z-axis actuator 982 causes the scanning assembly 400 to be positioned at minimum distance from the pupil of the patient 402. The y-axis actuator 986 is retracted to minimum height. The mirror 432 is rotated at a 35 degree angle relative to the focal plane to the eye 402.

FIG. 11C shows the vertical positioning assembly set to the middle of the vertical range of zero degrees. In this position, the z-axis actuator 982 causes the scanning assembly 400 to be positioned at a middle distance from the pupil of the patient 402. The y-axis actuator 986 is set to a middle height. The mirror 432 is rotated at a 45 degree angle relative to the focal plane to the eye 402.

FIG. 12A shows the positions resulting from control of the z-axis actuator 982, x-axis actuator 984 of the instrument 900 that positions the scanning assembly 400 on the positive 30 degree extreme of the horizontal range. When scanning over the horizontal range, the y-axis actuator 986 is fixed in position and the z-axis actuator 982 and the x-axis actuator 984 are positioned in coordination with rotating the mirror 432 via the rotational actuator 926. In this example, the z-axis actuator 982 causes the scanning assembly 400 to be positioned at minimum distance from the pupil of the patient 402. The y-axis actuator 986 is retracted to minimum height. The mirror 432 is rotated by the rotational actuator 926 at a 20 degree angle relative to the focal plane to the eye 402.

FIG. 12B shows the negative 30 degree extreme of the horizontal range of positioning the scanning assembly 400. In this position, the z-axis actuator 984 causes the scanning assembly 400 to be positioned at minimum distance from the pupil of the patient 402. The x-axis actuator 984 is moved to one end to offset the position of the scanning assembly 400. The mirror 432 is rotated by the rotational actuator 926 at a negative 20 degree angle relative to the focal plane to the eye 402.

FIG. 12C shows the horizontal scan system 912 set to the middle of the horizontal range of zero degrees. In this position, the z-axis actuator 982 causes the scanning assembly 400 to be positioned at a middle distance from the pupil of the patient 402. The x-axis actuator 984 is set to a middle position. The mirror 432 is rotated to a 0 degree angle relative to the focal plane to the eye 402 by the rotational actuator 926.

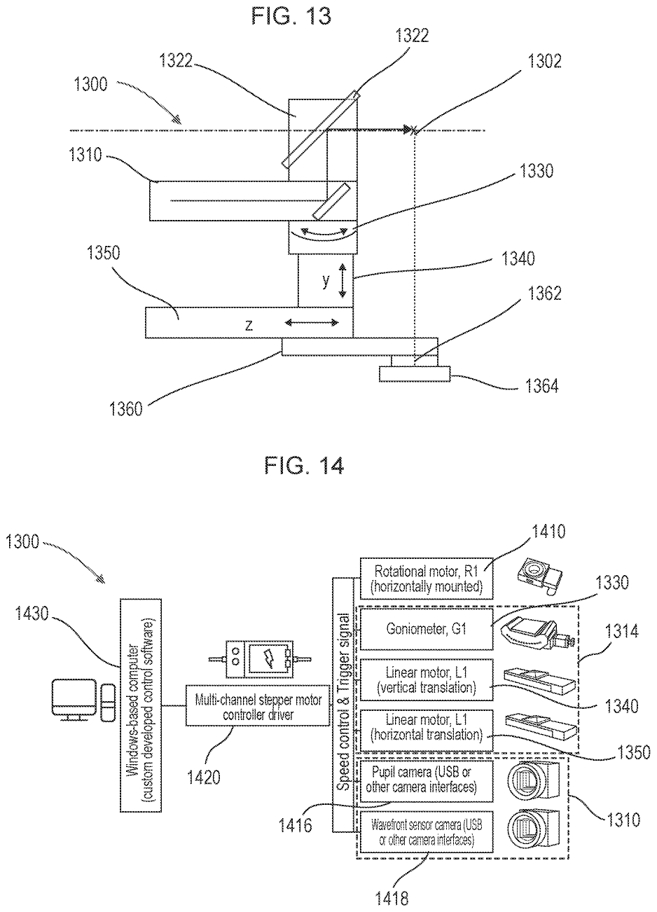

FIG. 13 shows a side view of another scanning instrument 1300 that positions a scanning assembly 1310 that is similar to the scanning assembly 400 in FIG. 4 relative to the eye of a patient 1302. The scanning assembly 1310 includes sensors, lens, cold mirrors, beam splitters and a laser to generate an optical signal to illuminate the eye 1302. The scanning instrument 1300 includes a horizontal scan movement system 1312 and a vertical scan movement system 1314 that allow positioning of the scanning assembly 1310 relative to the eye of the patient 1302. The scanning instrument 1300 includes a hot mirror 1320 fixed on a mirror support 1322 at a 45 degree angle. The mirror support 1322 is attached to a goniometer 1330 and the scanning assembly 1310. The goniometer 1330 allows the mirror 1320 to be angled relative to the focus axis of the patient 1302 based on the angle position of the goniometer 1330.

The goniometer 1330 and the scanning assembly 1310 are supported by a y-axis actuator 1340 that raises or lowers the goniometer 1330 and the scanning assembly 1310 relative to the focus axis of the eye 1302. The y-axis actuator 1340 is mounted on a z-axis actuator 1350 that may be moved longitudinally on an adaptor plate 1360. The adaptor plate 1360 is attached to a rotational table 1362 that may rotate the adaptor plate 1360. The rotational table 1362 is fixed to a base 1364. The use of the goniometer 1330 to rotate the scanning assembly 1310 and the mirror support 1322 allows for a more compact footprint of the instrument 1300.

In the example scanning instrument 1300, the horizontal movement system 1312 is the rotational table 1362 that may rotate the adaptor plate 1360 to position the scanning assembly 1310 relative to the patient 1302 in a horizontal range. The vertical movement system 1314 includes the y-axis actuator 1340, the z-axis actuator 1350, and the goniometer 1330 that may each be positioned to position the scanning assembly 1310 relative to the patient in a vertical range.

FIG. 14 shows a block diagram of the control system for the scanning system 1300 in FIG. 13. In this example, the horizontal scan system 1312 includes a rotational motor 1410 that rotates the rotational table 1352. The vertical scan system 1314 includes the z-axis actuator 1350, the y-axis actuator 1340, and the goniometer 1330 that rotates the mirror support 1322. A controller 1420 generates commands for operation of the individual motors in the actuators. Control of these motors by the controller 1420 allows for horizontal and vertical scanning by the scanning assembly 1310, while any diagonal meridian scan can be achieved by precisely synchronizing and combining the movement of both the horizontal scan system 1312 and the vertical scan system 1314. In this example, the control signals are generated by a personal computer 1430 with control software. The personal computer 1430 is coupled to the controller 1420 that is a multi-channel stepper motor controller driver. The controller 1420 provides trigger signals to the motors to adjust the angle of the scanning assembly 1310 in multiple dimensions for the scan patterns. Over each of the ranges, the controller 1310 activates a CCD image sensor 1316 and the SH wavefront sensor 1318 to capture sensed data from the eye over the scan ranges.

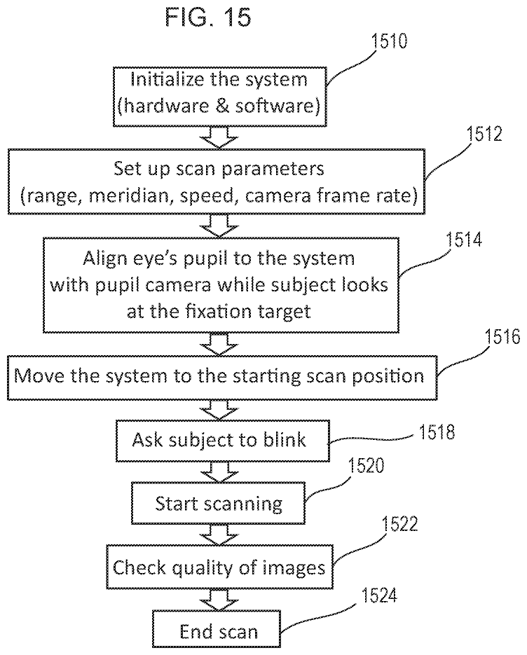

The flow diagram in FIG. 15 is representative of example machine readable instructions for the process of controlling the example scanning instruments to take a scan set of an eye. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowchart illustrated in FIG. 15, persons of ordinary skill in the art will readily appreciate that many other methods of implementing the example machine readable instructions may alternatively be used. For example, the order of execution of the blocks may be changed, and/or some of the blocks described may be changed, eliminated, or combined.

The routine begins by initializing the hardware and software of the instrument (1510). The scan parameters including range, meridian, speed and camera frame rate for the image sensor and the wavefront sensor are set (1512). The patient is then instructed to look at the fixation target while the pupil is aligned with the pupil camera (1514). The control system moves the horizontal and vertical scan systems to a starting scan position (1516). The patient is instructed to blink (1518). The scan process then begins by moving the actuators over the dimensional range and taking a series of wavefront images and pupil images (1520). As the number of desired images is collected, the quality of the captured images is checked (1522). The scan for the dimension is then ended (1524). In this example, the steps 1512 to 1524 are repeated for the vertical scan range, the horizontal scan range, and both diagonal scan ranges. Additional diagonal scan ranges could be taken by adjusting the controls for the actuators.

Testing of the example instrument in FIG. 2 was conducted. A simplified model eye was created to assess the precision of the scanning assembly 100 when evaluating peripheral aberrations. The model consisted of a doublet lens with a focal length of 30 mm (AC-254-030A from Thorlabs, Inc.) representing the cornea and lens of the eye, and a diffuser located at the focal point of the lens, mimicking the retina. Peripheral aberrations of the model eye, between 10 and 30 degrees of eccentricity in 5-degree increments, were measured using the example scanning assembly 100. To validate the measured results, an optical ray tracing program (Zemax, LLC) was used to simulate the model eye by using the exact lens parameters provided by the manufacturer.

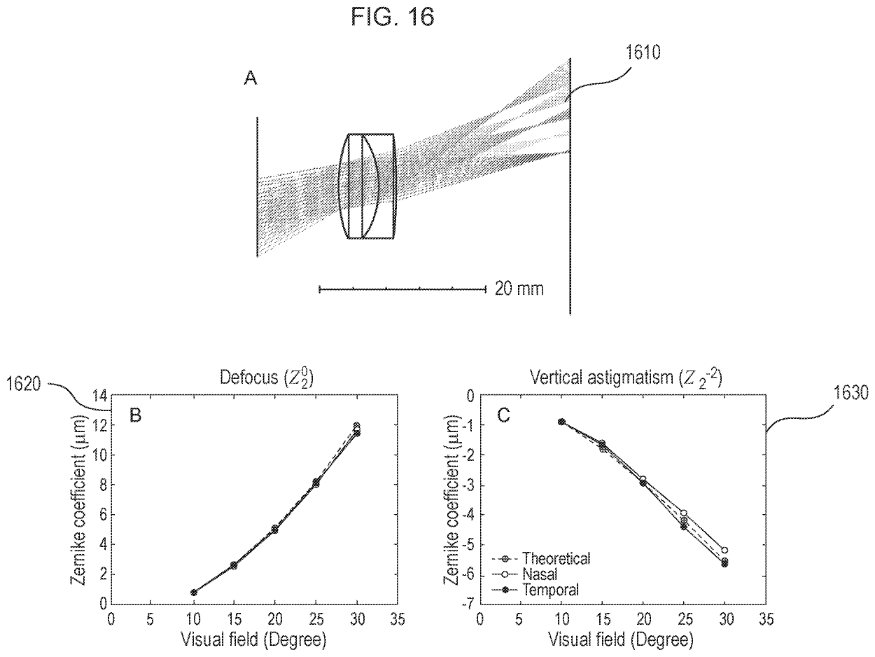

FIG. 16 shows data that validates the example scanning wavefront sensor by comparing theoretical peripheral aberrations with model eye data. The theoretical peripheral aberration was determined by Zemax ray-tracing as shown in the graph 1610 through a doublet lens (f=30 mm; AC-254-030A from Thorlabs, Inc.) with the exit pupil positioned on the lens back surface. The aberration was then measured with the WSWS for a 5.5 mm pupil. FIG. 16 shows graphs 1620 and 1630 that illustrate the theoretical and measured peripheral defocus and astigmatism of nasal and temporal model eye, respectively. The Y-axis represents Zernike coefficient in microns and has varying scaling to highlight the validation comparison.

Results from both the simulation and measurements were analyzed using a 5.5 mm pupil. For simplicity, a flat diffuser unlike the curved retina in the real eye was chosen, which resulted in large defocus at the peripheral visual fields (up to 11.95 μm at 30°). Both simulation and model eye measurements indicated that defocus and astigmatism were the most significant aberrations, as plotted in the graphs 1620 and 1620. The nasal and temporal defocus

$$( {Z}_{2}^{0} )$$

and vertical astigmatism

$$( {Z}_{2}^{- 2} )$$

showed good agreement with the theoretical values, especially closer to fovea. However, there was a small deviation that incur with eccentricity (max deviation:

$$( {Z}_{2}^{0} )$$

=0.36 μm and

$$( {Z}_{2}^{- 2} ) = 0.13 μ m$$

respectively at 30° visual field).

Peripheral scans were conducted in all 22 participants along the horizontal (±30°) and vertical meridians (±18°), while oblique scans (±18°) were only measured in eight subjects. On-eye peripheral aberrations were calculated across the scanning range using a 5.5 mm pupil.

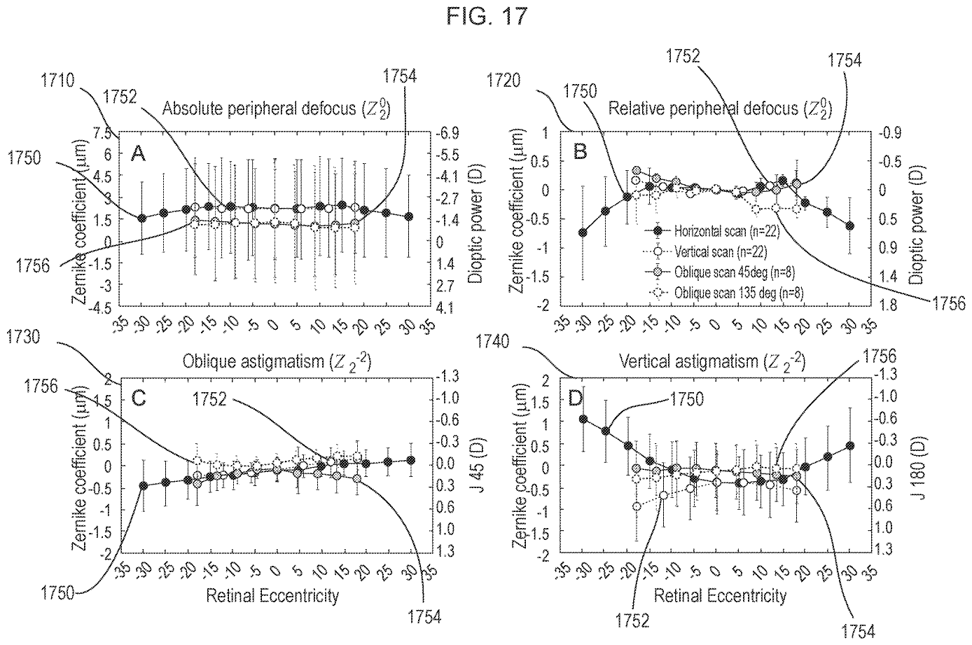

FIG. 17 shows a series of graphs 1710, 1720, 1730, and 1740 that show Peripheral lower-order aberrations (LOAs) plotting Zernike coefficient against retinal eccentricity. The graph 1710 shows defocus

$$( {Z}_{2}^{0} ) ;$$

the graph 1720 snows defocus relative to the fovea; the graph 1730 shows oblique astigmatism

$$( {Z}_{2}^{- 2} ) ;$$

and the graph 1740 shows vertical astigmatism

$$( {Z}_{2}^{2} ) .$$

The measured aberrations are plotted at four distinct retinal eccentricities: a horizontal (line 1750, n=22) scan covering±30° from the fovea, a vertical (line 1752, n=22) scan, and two oblique scans (n=8) along 45° (line 1754) and 135° (line 1756), each covering±18° from the fovea. The X-axis for each of the graphs 1710, 1720, 1730, and 1740 displays the degree of retinal eccentricity, with negative values indicating temporal, inferior, infero-temporal, and infero-nasal retinal eccentricities for horizontal, vertical, diagonal 45°, and diagonal 135° scans, respectively. The Y-axis for each of the graphs 1710, 1720, 1730, and 1740 displays the aberration magnitude in microns, while the secondary Y-axis represents the equivalent dioptric power of defocus and astigmatic components (J0 & J45) for a 5.5 mm pupil. The amplitude scales in graphs 1710 and 1720 differ to better illustrate the absolute and relative defocus distribution across retinal eccentricities. The error bars represent the standard deviation at each eccentricity.

The graphs 1710, 1720, 1730, and 1740 depict the lower-order aberrations (LOAs), which indicate defocus

$$( {Z}_{2}^{0} )$$

and astigmatism

$$( {Z}_{- 2}^{2} \& {Z}_{2}^{2} )$$

components across the four scanning directions. The standard deviation error bars represent inter-subject variability at each eccentric point. In the sample, the mean on-axis defocus at the fovea was 2.28±3.31 μm. A slight deviation was noticed between foveal defocus values obtained from horizontal and vertical scans (0.12±0.15 μm) and between oblique scans (0.27±0.34 μm). The relatively small deviation to pupil decentration was attributed due to slight variations in the subjects' fixation during the different scans (as shown in the graph 1710). The average foveal value in oblique scans varies from that in horizontal and vertical scans, primarily because of the difference in sample size.

To show the change in peripheral defocus relative to the foveal value, the foveal value was subtracted from each peripheral measurement point and the results were presented in the graph 1720, where error bars represent the variance of peripheral defocus from the foveal value at each tested eccentricity. There was a gradual increase in relative hyperopic defocus towards both the temporal (−0.61±0.48 μm at 30°) and nasal (−0.73±0.80 μm at 30°) periphery with increasing horizontal eccentricity. Conversely, across the vertical meridian, relative myopic defocus gradually increased towards the inferior (0.12±0.20 μm at 18°) and superior (0.16±0.07 μm at 18°) periphery. The results from the oblique scans (n=8) demonstrated a trend of relative peripheral defocus shift between 45° and 135° meridians. Specifically, the oblique 45° scan showed a gradual increase in relative myopic defocus towards the periphery (0.10±0.42 μm at 18° infero-nasal and 0.32±0.03 μm at 18° supero-temporal retina), while the 135° scan exhibited relative hyperopic defocus towards the periphery (−0.32±0.15 μm at 18° infero-temporal and −0.09±0.49 μm at 18° supero-nasal retina).

The test data indicates negative vertical astigmatism

$$( {Z}_{2}^{2} )$$

on-axis (−0.39±0.63 μm), which gradually changes to positive

$${Z}_{2}^{2}$$

towards the temporal anu nasal eccentricities (0.45±0.86 μm and 1.05±0.75 μm, respectively). Interestingly, along the vertical meridian

$${Z}_{2}^{2}$$

showed an opposite trend, becoming more negative towards the inferior (−0.56±0.75 μm) and superior (−0.93±0.79 μm) retina. The oblique scans showed a combination of oblique astigmatism

$$( {Z}_{- 2}^{2} ) and {Z}_{2}^{2}$$

distribution. The slightly negative on-axis astigmatisms

$$( {Z}_{- 2}^{2} = - 0.10 \pm 0.27 μm and {Z}_{2}^{2} = - 0 \text{.16} \pm 0.61 μm )$$

gradually increased in magnitude to −0.30±0.38 μm and −0.25±0.62 μm

$$( {Z}_{- 2}^{2} \& {Z}_{2}^{2} at 18 ° inferno - nasal retina , respectively )$$

and −0.43±0.50 μm and −0.08±0.58 μm

$$( {Z}_{- 2}^{2} \& {Z}_{2}^{2} at 18 ° supero - temporal retina , respectively )$$

across the 45° meridional scan. While

$${Z}_{2}^{2}$$

maintained a similar trend along the oblique scan,

$${Z}_{- 2}^{2}$$

showed an opposite trend of slightly increasing magnitude at the periphery (relative change from fovea: 0.06±0.15 μm and 0.16±0.09 μm at infero-temporal and supero-nasal 18° retina, respectively). In general, defocus demonstrated a more symmetrical distribution between two eccentricities of each scanning meridian, whereas vertical astigmatism displayed a similar tendency but with a slight asymmetry.

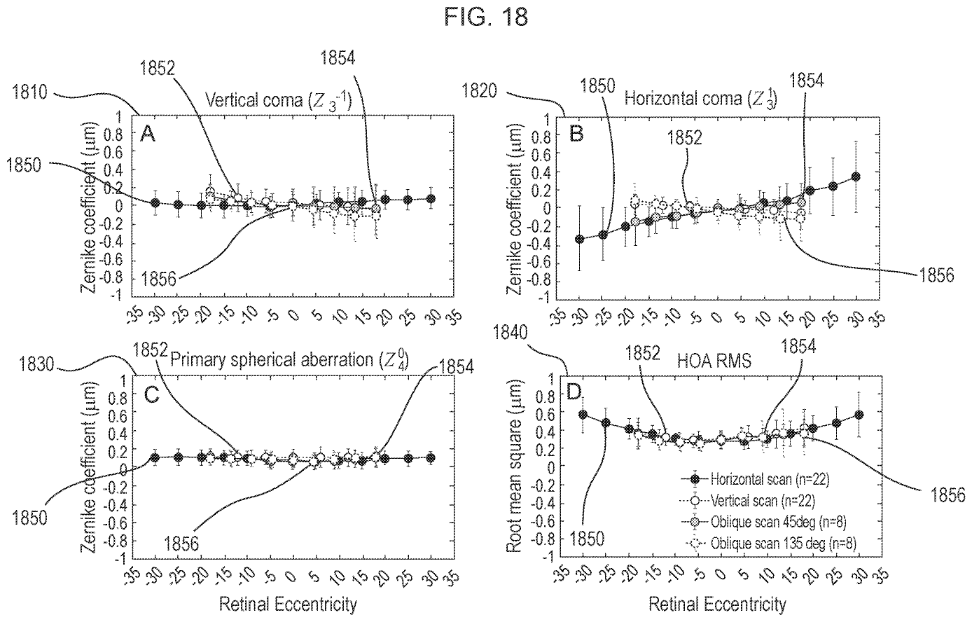

FIG. 18 shows a series of graphs 1810, 1820, 1830 and 1840 that show higher-order aberrations (HOAs) across the retinal eccentricity. The graph 1810 shows vertical coma

$$( {Z}_{3}^{- 1} ) ;$$

the graph 1820 shows vertical astigmatism

$$( {Z}_{3}^{1} ) ;$$

the graph 1830 shows primary spherical aberration

$$( {Z}_{4}^{0} ) ;$$

and the graph 1840 shows HOA root-mean-square (RMS). The measured aberrations are plotted at four distinct retinal eccentricities: a horizontal (line 1850, n=22) scan covering±30° from the fovea, a vertical (line 1852, n=22) scan, and two oblique scans (n=8) along 45° (line 1854) and 135° (line 1856), each covering±18° from the fovea. The X-axis of each of the graphs 1810, 1820, 1830, and 1840 display retinal eccentricity in degrees, with negative values indicating temporal, inferior, infero-temporal, and infero-nasal retinal eccentricities for horizontal, vertical, diagonal 45°, and diagonal 135° scans, respectively. The Y-axis of each of the graphs 1810, 1820, 1830, and 1840 represents the magnitude of aberrations in microns, calculated for a 5.5 mm pupil size. The error bars represent the standard deviation at each eccentric point.

In the graphs 1810, 1820, 1830, and 1840, the root mean square (RMS) of HOAs and their primary contributors, including vertical

$$( {Z}_{3}^{- 1} )$$

and horizontal coma

$$( {Z}_{3}^{1} )$$

as well as primary spherical aberration

$$( {Z}_{4}^{0} ) ,$$

are shown. Both the coma components exhibit an asymmetric distribution across retinal eccentricities, with

$${Z}_{3}^{1}$$

being dominant across the horizontal meridian and

$${Z}_{3}^{- 1}$$

across the vertical meridian. The magnitude of

$${Z}_{3}^{1}$$

changes linearly from negative (−0.33±0.36 μm) to positive (0.35±0.40 μm) as retinal eccentricity increases from temporal to nasal 30°. The magnitude of

$${Z}_{3}^{- 1}$$

decreases gradually from 0.17±0.18 μm at inferior 18° eccentricity to −0.03±0.28 μm at superior 18° eccentricity.

The distribution of vertical coma also showed changes in the oblique scans towards the periphery, whereas there was negligible change in

$${Z}_{3}^{1} .$$

In the oblique 45° scan,

$${Z}_{3}^{- 1}$$

changed from a slightly negative magnitude to positive as it progressed from the 18° inferior to 18° superior meridians (−0.02±0.25 μm to 0.12±0.12 μm). Similarly, the oblique 135° scan exhibited a similar trend, with

$${Z}_{3}^{- 1}$$

changing from −0.10±0.27 μm to 0.08±0.14 μm as it progressed from the inferior to superior 18° eccentricities. The average on-axis primary spherical aberration was +0.08±0.08 μm and did not exhibit significant variation across peripheral eccentricities. The RMS magnitude of all HOAs increases gradually with eccentricity across all meridians.

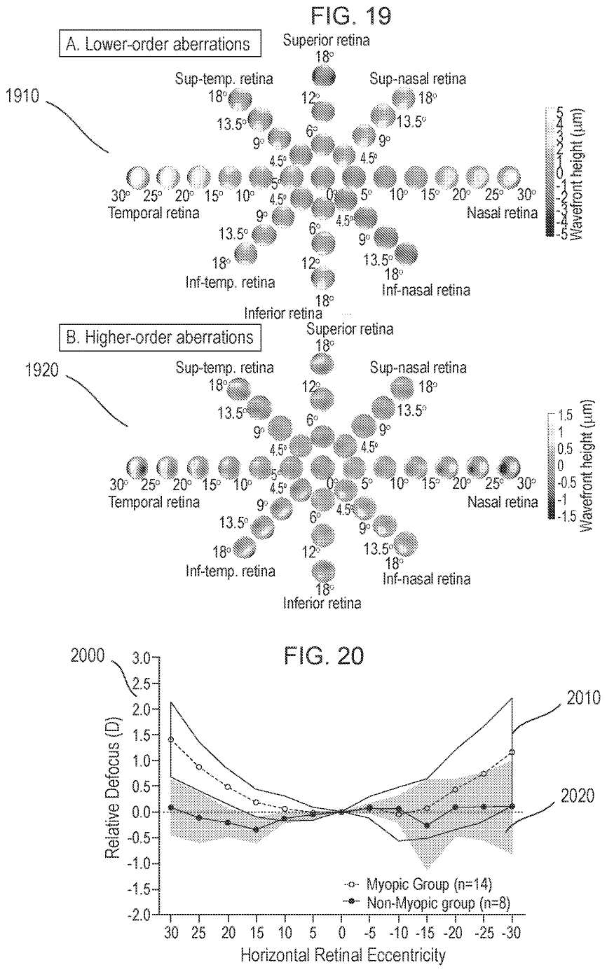

FIG. 19 shows two plots 1910 and 1920 that are pan-retinal schematic representations of wavefront maps reconstructed from the measured aberrations for a 5.5 mm pupil across all four scanning directions (vertical, horizontal, and two diagonals). The plot 1910 shows distribution of peripheral lower-order aberrations. Foveal defocus value is subtracted from each retinal location. The plot 1920 shows the distribution of higher-order aberrations. The colormap shows the amplitude of wavefront height in μm.

The plots 1910 and 1920 are wavefront maps that were reconstructed using measured Zernike coefficients for a 5.5 mm pupil across various data points of the wavefront sensor output from one of the participants in the study. Overall, the aberration distribution across the retina indicated an increasing magnitude of aberrations and a progressively rotationally asymmetric aberration profile with greater retinal eccentricity. The plot 1910 depicts the distribution of peripheral defocus and astigmatism across the measured retinal fields. The horizontal peripheral eccentricity resulted in a more significant shift in astigmatism and defocus than other meridians due to the larger scanning range across the nasal and temporal eccentricities. The plot 1920 shows the distribution of HOAs, which revealed asymmetric aberration patterns, particularly across the horizontal meridian and inferior retinal locations. These trends were consistent in other study participants with the mean results presented in the graphs in FIG. 18, indicating a trend of negative to positive horizontal coma across the horizontal meridian from temporal to nasal retina. The images from the pupil camera that correspond to each of the wavefront sensor images may be used to validate the analysis and alignment of the instrument relative to the eye.

The example novel wavefront sensor is capable of scanning a wide range of eccentricities across any retinal meridian in less than 5 seconds. The multi-directionality of the example instruments is attained by way of a unique scanning method that involves the concurrent operation of horizontal and vertical scan systems and allows measurement of a wide retinal meridian within a single blink interval. Additionally, the example instruments are designed for natural eye fixation at the primary gaze, rather than relying on off-axis fixation to align the system to the subject's eye. Tests show the successful demonstration of measuring aberrations of human eyes along the horizontal, vertical, and two oblique meridians.

The performance of the example scanning instrument has been validated and on-eye aberration measurement was performed on 22 young individuals, having different spherical refractive errors (mean±stdev: −1.98±3.06 D). The results indicated a gradual decrease in the defocus component towards the temporal and nasal periphery, reflecting a relative peripheral hyperopic shift. To better understand the peripheral relative defocus distribution, the subjects were grouped into myopic (−3.74±2.30 D, n=14) and non-myopic (−0.03±1.76 D, n=8) categories based on their foveal refraction. FIG. 20 is a graph 2000 that shows relative defocus (D) from the fovea across horizontal meridian in myopic (represented by an area 2010) and non-myopic (represented by area 2020) groups. Error bars indicated the standard deviation. The graph 2000 shows the myopic group having relative peripheral hyperopia, whereas non-myopic subjects revealed mean relative peripheral near-emmetropic defocus.

Consistent with the opposite defocus distribution observed along the vertical and oblique meridians (shown in FIGS. 17 and 19), previous studies have reported a pan-retinal defocus distribution with a contrasting trend along different meridians, which is in line with our results from the vertical and oblique scans. In addition to defocus, a trend of bidirectional change of vertical astigmatism along the horizontal and vertical eccentricities was observed. Previous studies have reported similar distributions of astigmatism along the horizontal and vertical meridians. Furthermore, a similar trend in oblique astigmatism along the diagonal meridians was observed.

Among the study participants, the root mean square (RMS) of HOAs increased as retinal eccentricity increased. This is mainly due to the influence of vertical coma

$$( {Z}_{3}^{- 1} ) ,$$

horizontal coma

$$( {Z}_{3}^{1} ) .$$

The analysis of horizontal scans indicated a linear change in

$${Z}_{3}^{1}$$

magnitude, ranging from negative to positive values across temporal to nasal retinal eccentricity as shown in the graph 1820 in FIG. 18. Meanwhile, the vertical meridional scan revealed that

$${Z}_{3}^{- 1}$$

was the dominant component, changing from a positive to negative coefficient from inferior to superior retinal locations. This observation is well documented, especially across the horizontal and vertical meridians. The average on-axis

$${Z}_{4}^{0}$$

reported as 0.14 μm for 5.8 mm pupil in a large-population study, which is close to the on-axis mean

$${Z}_{4}^{0}$$

in the study participants (+0.08±0.08 μm, measured over 5.5 mm pupil). The

$${Z}_{4}^{0}$$

term exhibits minimal change across the peripheral retinal locations, which is supported by earlier reports but limited to horizontal and vertical eccentricities. The consistency of obtained LOA and HOA across the measured field with relevant literature proves efficacy of the on-eye performance by the example instrument.

The example wavefront scanning instruments overcome limitations in current peripheral wavefront measurements. This capability expands the applications of aberration quantification to include contact lens-induced peripheral aberration assessment, which is essential for development of more effective corrective lenses for myopia control. The usage of primary gaze fixation is crucial in this discipline to avoid unwanted contact lens decentration with eccentric fixations. Furthermore, natural primary fixation allows for a more comfortable measurement procedure, from the subject's perspective, which may allow the inclusion of subjects such as children. Quantifying peripheral aberrations in children is crucial in gaining a better understanding of how ocular optics contribute to emmetropization and ocular development.

Additional features may be added. Corneal reflection of the light source from the anterior corneal surface at scanning angles close to on-axis caused interference with Zernike analysis. This can be avoided by slightly dislocating the laser beam from the corneal apex, however, that may also cause a similarly sized offset between expected and actual scanning angle. The optical design could further be improved with the implementation of a longer wavelength light source that is completely invisible to the human eye. The current design uses near infrared light which is still visible and may interrupt fixation during scanning. Another limitation is the portability of the device. A compact portable peripheral wavefront sensing device could expand possible applications of the WSWS to include high throughput data collection in clinics or schools. This kind of flexibility could also enable longitudinal experiments, which are critical for untangling the underlying mechanisms of visual-driven processes such as myopia development and progression. The scanning instruments could also mount any type of imaging sensor other than the disclosed wavefront and camera. For example, an optical computed tomography (CT) sensoroptical coherence tomography (OCT) or a partially coherent interferometer could be mounted on the scanning assembly to collect ocular biometry CT data such as axial length.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

Each of these embodiments and obvious variations thereof is contemplated as falling within the spirit and scope of the claimed invention, which is set forth in the following claims.

1. An imaging system for capturing images of the periphery of an eye of a patient, the system comprising:
an optical signal source providing an optical source light;
a microlens array placed at the pupil conjugate of the patient;
an image sensor receiving focused light from the microlens array;
a horizontal scan system coupled to the microlens array and the image sensor to capture images in a scan range of angles in the horizontal plane to the eye;
a vertical scan system coupled to the microlens array and the image sensor to capture images in a scan range of vertical angles relative to a horizontal plane to the eye; and
a controller coupled to the horizontal scan system and the vertical scan system, the controller activating the horizontal scan system to capture images of the eye in a horizontal scan; the controller activating the vertical scan system to capture images of the eye in a vertical scan, and the controller activating both the horizontal scan system and the vertical scan system to capture images of the eye in a diagonal scan.

2. The system of claim 1, wherein the horizontal scan system includes a rotational table supporting the micro-lens array and image sensor, the rotational table rotated by a first actuator.

3. The system of claim 1, wherein the horizontal scan system includes a rotatable hot mirror directing the optical source light to the eye, a first linear actuator moving in a first dimension, and a second linear actuator coupled to the first linear actuator moving in a second dimension perpendicular to the first dimension, wherein the micro-lens array and image sensor are coupled to the second linear actuator, wherein the first linear actuator, the second linear actuator and the rotatable hot mirror are controlled in the scan range of angles in the horizontal plane.

4. The system of claim 1, wherein the vertical scan system includes a hot mirror directing the optical source light to the eye, a first linear actuator moving in a first dimension, and a second linear actuator coupled to the first linear actuator moving in a second dimension perpendicular to the first dimension, wherein the micro-lens array and image sensor are coupled to the second linear actuator, wherein the first linear actuator, the second linear actuator and the rotatable hot mirror are controlled in the scan range of angles in vertical horizontal plane.

5. The system of claim 4, wherein the hot mirror is rotated via a belt coupled to a motor.

6. The system of claim 4, wherein the patient maintains foveal fixation on the target object through the hot mirror, and wherein the hot mirror is a rectangular shape.

7. The system of claim 4, further comprising a goniometer coupled to hot mirror to rotate the hot mirror.

8. The system of claim 1, further comprising:
a target object; and
a cold mirror allowing the patient to focus on the target object.

9. The system of claim 8, wherein the vertical scan system is a goniometer, and wherein the goniometer swings the micro-lens array and wavefront image sensor in the vertical plane relative to the eye.

10. The system of claim 1, wherein the image sensor is activated by the controller to provide real-time images at predetermined degree ranges in the horizontal, vertical and diagonal retinal eccentricities.

11. The system of claim 1, wherein the image sensor takes a plurality of images along predetermined degree increments in the horizontal, vertical, and diagonal ranges.

12. The system of claim 1, wherein the imaging sensor is a high sensitivity charge coupled device (CCD) sensor.

13. The system of claim 1, wherein the imaging sensor is a Shack-Harmann wavefront imaging sensor.

14. The system of claim 13, further comprising an output beam splitter receiving the reflected light from the eye through the microlens array and splitting the reflected light to the imaging sensor.

15. The system of claim 14, further comprising a pupil camera accepting the reflected light from the output beam splitter to monitor eye movements of the patient.

16. The system of claim 1, wherein the optical source is a laser diode.

17. The system of claim 1, further comprising a ring illuminator positioned in proximity to the eye, wherein the pupil is illuminated via the ring illuminator.

18. An imaging system for capturing images of the periphery of an eye of a patient, the system comprising:
an optical signal source providing an optical source light;
a microlens array placed at the pupil conjugate of the patient;
a wavefront image sensor receiving focused light from the microlens array;
a rotational table coupled to the microlens array and the wavefront image sensor to rotate the microlens array and the wavefront image sensor capture wavefront images in a scan range of angles in the horizontal plane to the eye;
a vertical scan system mounted on the rotational table to capture wavefront images in a scan range of vertical angles relative to a horizontal plane to the eye, wherein the vertical scan system includes a hot mirror, a goniometer supporting the hot mirror to direct the optical source light to the eye, a first linear actuator moving in a first dimension, and a second linear actuator coupled to the first linear actuator moving in a second dimension perpendicular to the first dimension, wherein the goniometer, the micro-lens array and wavefront image sensor are coupled to the second linear actuator, and wherein the first linear actuator, the second linear actuator and the goniometer are controlled in the scan range of vertical angles relative to a horizontal plane to the eye; and
a controller coupled to the horizontal scan system and the vertical scan system, the controller activating the horizontal scan system to capture wavefront images of the eye in a horizontal scan; the controller activating the vertical scan system to capture wavefront images of the eye in a vertical scan, and the controller activating both the horizontal scan system and the vertical scan system to capture wavefront images of the eye in a diagonal scan.

19. A method for capturing images for detection of aberrations in an eye, the method comprising:
providing an optical source light at the pupil conjugate of the eye;
capturing images of the eye via a microlens array over a horizontal range of angles in the horizontal plane to the eye by positioning an image sensor coupled to a microlens array via a horizontal scan system over the horizontal range;
capturing images of the eye via the microlens array over a vertical range of angles in the horizontal plane to the eye by positioning the image sensor coupled to the microlens array via a vertical scan system over the vertical range; and
capturing images of the eye via the microlens array over a diagonal range of angles in the horizontal plane to the eye by positioning the image sensor coupled to the microlens array via both the horizontal scan system and the vertical scan system over the diagonal range.
